(19) United States
(12) Patent Application Publication
(10) Pub. No.: US 2022/0257629 A1
(43) Pub. Date: Aug. 18, 2022

(54) COMPOSITIONS AND METHODS FOR UPREGULATION OF HUMAN FETAL HEMOGLOBIN

(71) Applicant: THE UAB RESEARCH FOUNDATION, Birmingham, AL (US)

(72) Inventors: Tim Townes, Birmingham, AL (US); Dewang Zhou, Birmingham, AL (US)

(21) Appl. No.: 17/630,133

(22) PCT Filed: Jul. 30, 2020
(86) PCT No.: PCT/US2020/044185

§ 371 Date: Jan. 25, 2022

Related U.S. Application Data
(60) Provisional application No. 62/880,447, filed on Jul. 30, 2019.

(51) Int. Cl. A61K 31/7072; A61P 7/06

(52) U.S. Cl. CPC A61K 31/7072; A61P 7/06

(57) ABSTRACT

Provided herein are compositions and methods for increasing fetal hemoglobin in a subject in need thereof. Also provided are compositions and methods for treating a hemoglobin disorder in a subject.

### PRIOR RELATED APPLICATION

This application claims the benefit of U.S. Provisional Application No. 62/880,447 filed on Jul. 30, 2019, which is hereby incorporated by reference in its entirety.

### STATEMENT REGARDING FEDERALLY FUNDED RESEARCH

This invention was made with Government Support under DK073391 awarded by the National Institutes of Health. The government has certain rights in the invention.

### BACKGROUND

Hemoglobin disorders affect millions of people worldwide. For example, the World Health Organization (WHO) estimates that, each year, 200,000 infants are born with sickle cell anemia in Africa. A majority of these infants will die from anemia and infections before they are five years old. In the United States, there are about 80,000 to 100,000 individuals with sickle cell anemia. Current treatments, for example, stem cell transplants and transfusions, have substantial risks.

### SUMMARY OF THE INVENTION

Provided are methods for increasing the amount of fetal hemoglobin (HbF) in the blood of a subject. The methods comprise administering to a subject in need thereof an effective amount of a compound having Formula I:

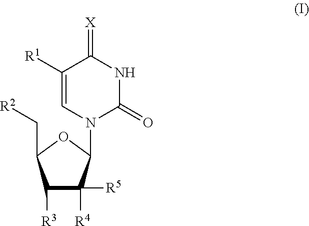

or a prodrug thereof, or a pharmaceutically acceptable salt thereof, wherein R¹, R² and R³ are independently a (a) halogen atom; (b) a hydroxyl group; (c) (d) an amino group; (e) a sulfhydryl group; (e) a nitro group; (f) an azido group; (g) a cyano group; (h) an ethenyl group; (i) an ethynyl group; (j) an aromatic or non-aromatic heterocyclic group; (k) an aryl group, wherein hydrogen atoms in (h), (i), (j), and (k) are optionally substituted with halogen, hydroxyl, nitro, azido, cyano, amino, sulfhydryl, phenyl, ethenyl, ethynyl, or an aromatic/non-aromatic heterocyclic group, and wherein the hydrogen atoms in the said phenyl, ethenyl, ethynyl, or aromatic/non-aromatic heterocyclic group are optionally substituted with halogen, hydroxyl, nitro, azido, cyano, amino, or sulfhydryl; (l) methyl substituted with halogen, hydroxyl, nitro, azido, cyano, amino, sulfhydryl, phenyl, ethenyl, ethynyl, or an aromatic/non-aromatic heterocyclic group, and wherein the hydrogen atoms in the said phenyl, ethenyl, ethynyl, and aromatic/non-aromatic heterocyclic group are optionally substituted with halogen, hydroxyl, nitro, azido, cyano, amino, or sulfhydryl; or (m) oxygen substituted with C₁-C₂₀ alkyl, C₁-C₂₀ acyl, C₁-C₂₀ alkoxycarbonyl, or carbamoyl; X is O or S; Y is H, O, S, or N; and R⁴ and R⁵ are independently H, halogen, or C₁-C₂₀ alkyl.

In some examples, the compound has Formula II:

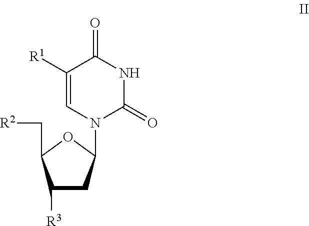

or a prodrug thereof, or a pharmaceutically acceptable salt or ester of said compound or prodrug, wherein R¹, R² and R³ are independently (a) a halogen atom; (b) a hydroxyl group; (c) an amino group; (d) a sulfhydryl group; (e) a nitro group; (f) an azido group; (g) a cyano group; (h) an ethenyl group; (i) an ethynyl group; (j) an aromatic/non-aromatic heterocyclic group; (k) an aryl group, wherein hydrogen atoms in (h), (i), (j) and (k) are optionally substituted with halogen, hydroxyl, nitro, azido, cyano, amino, sulfhydryl, phenyl, ethenyl, ethynyl, or an aromatic/non-aromatic heterocyclic group, and wherein the hydrogen atoms in the said phenyl, ethenyl, ethynyl, or aromatic/non-aromatic heterocyclic group are optionally substituted with halogen, hydroxyl, nitro, azido, cyano, amino, or sulfhydryl; (l) methyl substituted with halogen, hydroxyl, nitro, azido, cyano, amino, sulfhydryl, phenyl, ethenyl, ethynyl, or an aromatic/non-aromatic heterocyclic group, and wherein the hydrogen atoms in the said phenyl, ethenyl, ethynyl, and aromatic/non-aromatic heterocyclic group are optionally substituted with halogen, hydroxyl, nitro, azido, cyano, amino, or sulfhydryl; or (m) oxygen substituted with Ci-Cao alkyl, Ci-Cao acyl, Ci-Cao alkoxycarbonyl, or carbamoyl.

In some examples, idoxuridine, trifluridine or a pharmaceutically acceptable salt thereof is administered to the subject. In some examples, a prodrug of Formula I or Formula II, for example, an idoxuridine prodrug is administered to the subject

Also provided are methods for treating a subject with a blood disorder associated with a defect in hemoglobin producing red blood cells or in the production of hemoglobin. The methods comprise administering to the subject with the blood disorder an effective amount of a compound having Formula I or Formula II, as defined above. In some examples, idoxuridine, an idoxuridine prodrug, trifluridine or a pharmaceutically acceptable salt thereof is administered to the subject.

Further provided herein are pharmaceutical compositions for non-topical administration of a compound having Formula I or Formula II or a prodrug thereof. In some examples, the pharmaceutical compositions comprise idoxuridine, an idoxuridine prodrug, trifluridine or a pharmaceutically acceptable salt thereof. Also provided are pharmaceutical compositions comprising a compound having Formula I, Formula II or a prodrug thereof, for example, idoxuridine, an idoxuridine prodrug, trifluridine or a pharmaceutically acceptable salt thereof, in combination with a second therapeutic agent (e.g., hydroxyurea, a histone deacetylase inhibitor, a lysine-specific histone demethylase 1 inhibitor, or a G9a methyltransferase inhibitor).

Further provided are packages or kits comprising one or more unit doses of a compound having Formula I, Formula II or a prodrug thereof, for example, idoxuridine, an idoxuridine prodrug, trifluridine or a pharmaceutically acceptable salt of idoxuridine, a pharmaceutically acceptable salt of an idoxuridine prodrug, or a pharmaceutically acceptable salt of trifluridine, optionally in combination with a second therapeutic agent.

### BRIEF DESCRIPTION OF THE FIGURES

FIG. 1 shows FACS analysis of erythroid cells treated with DMSO (control) or idoxuridine (0.3, 0.5, or 0.7 μM). Idoxuridine increases the number of erythroid cells expressing fetal hemoglobin (F cells) in human CD34⁺-differentiated erythroid cells as compared to a DMSO control.

FIG. 2 is an HPLC analysis showing that idoxuridine (0.3 or 0.65 μM) significantly increases HbF in human CD34⁺-differentiated erythroid cells as compared to DMSO treated cells.

FIG. 3 is an HPLC analysis showing that idoxuridine (0.5 μM) cooperates with hydroxyurea (10 μM) in upregulating HbF expression in human CD34⁺-differentiated erythroid cells.

FIG. 4 shows that, when samples analyzed by HPLC were run on an isoelectric focusing gel, idoxuridine (0.3 and 0.65 μM) upregulated HbF in human CD34⁺-differentiated erythroid cells, and idoxuridine (0.5 μM) cooperated with hydroxyurea (10 μM) in upregulating HbF expression in human CD34⁺-differentiated erythroid cells.

FIG. 5 is a Western blot showing that idoxuridine decreases KLF1 and BCL11A expression in human CD34⁺-differentiated erythroid cells as compared to expression in cells treated with DMSO.

FIG. 6 shows that, after daily intraperitoneal injections of idoxuridine for four weeks, idoxuridine increased mouse Ey-2 and human γ-globin gene expression in human β-globin BAC transgenic mice as compared to expression in cells treated with DMSO.

FIG. 7 shows that idoxuridine (labeled as Drug 1100) and trifluridine (labelled as Drug 1200) synergize to reactivate γ-globin gene expression in adult erythroid progenitor cells.

### DETAILED DESCRIPTION

Hemoglobin disorders, for example, sickle cell disease and β-thalassemia, can be alleviated or even cured by upregulation of HbF. Adult hemoglobin (HbA, α₂.β₂) is the most common human hemoglobin tetramer, accounting for over 97% of the total red blood cell hemoglobin in a subject. Fetal hemoglobin (HbF, α₂.γ₂) is the main oxygen transport protein in the human fetus during the last seven months of development in the uterus and persists in the newborn until roughly 2-4 months old. In newborns, fetal hemoglobin is almost completely replaced by adult hemoglobin once the infant is about six months old. Renewed or increased HbF expression is useful for the treatment of hemoglobin disorders. For example, and not to be limiting, the compositions and methods provided herein can be used to increase HbF expression in a subject, to reduce anemia in β-thalassemia or inhibit sickling in sickle cell disease.

Provided herein is a method for increasing the amount of HbF in the blood of a subject, comprising administering to a subject in need thereof an effective amount of one or more compounds having Formula I:

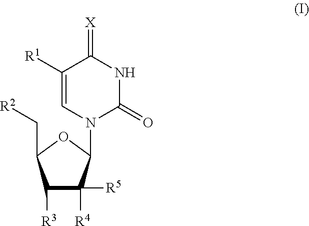

or a prodrug thereof, or a pharmaceutically acceptable salt thereof, wherein R¹, R² and R³ are independently a (a) halogen atom; (b) a hydroxyl group; (c) (d) an amino group; (e) a sulfhydryl group; (e) a nitro group; (f) an azido group; (g) a cyano group; (h) an ethenyl group; (i) an ethynyl group; (j) an aromatic or non-aromatic heterocyclic group; (k) an aryl group, wherein hydrogen atoms in (h), (i), (j), and (k) are optionally substituted with halogen, hydroxyl, nitro, azido, cyano, amino, sulfhydryl, phenyl, ethenyl, ethynyl, or an aromatic/non-aromatic heterocyclic group, and wherein the hydrogen atoms in the said phenyl, ethenyl, ethynyl, or aromatic/non-aromatic heterocyclic group are optionally substituted with halogen, hydroxyl, nitro, azido, cyano, amino, or sulfhydryl; (l) methyl substituted with halogen, hydroxyl, nitro, azido, cyano, amino, sulfhydryl, phenyl, ethenyl, ethynyl, or an aromatic/non-aromatic heterocyclic group, and wherein the hydrogen atoms in the said phenyl, ethenyl, ethynyl, and aromatic/non-aromatic heterocyclic group are optionally substituted with halogen, hydroxyl, nitro, azido, cyano, amino, or sulfhydryl; or (m) oxygen substituted with C₁-C₂₀ alkyl, C₁-C₂₀ acyl, C₁-C₂₀ alkoxycarbonyl, or carbamoyl; X is O or S; Y is H, O, S, or N; and R⁴ and R⁵ are independently H, halogen, or C₁-C₂₀ alkyl.

In some methods, the compound has Formula II:

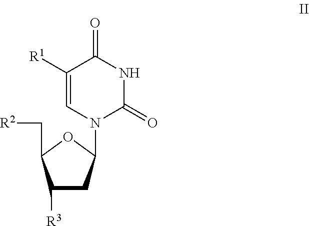

or a prodrug thereof, or a pharmaceutically acceptable salt or ester of said compound or prodrug, wherein R¹, R² and R³ are independently (a) a halogen atom; (b) a hydroxyl group; (c) an amino group; (d) a sulfhydryl group; (e) a nitro group; (f) an azido group; (g) a cyano group; (h) an ethenyl group; (i) an ethynyl group; (j) an aromatic/non-aromatic heterocyclic group; (k) an aryl group, wherein hydrogen atoms in (h), (i), (j) and (k) are optionally substituted with halogen, hydroxyl, nitro, azido, cyano, amino, sulfhydryl, phenyl, ethenyl, ethynyl, or an aromatic/non-aromatic heterocyclic group, and wherein the hydrogen atoms in the said phenyl, ethenyl, ethynyl, or aromatic/non-aromatic heterocyclic group are optionally substituted with halogen, hydroxyl, nitro, azido, cyano, amino, or sulfhydryl; (l) methyl substituted with halogen, hydroxyl, nitro, azido, cyano, amino, sulfhydryl, phenyl, ethenyl, ethynyl, or an aromatic/non-aromatic heterocyclic group, and wherein the hydrogen atoms in the said phenyl, ethenyl, ethynyl, and aromatic/non-aromatic heterocyclic group are optionally substituted with halogen, hydroxyl, nitro, azido, cyano, amino, or sulfhydryl; or (m) oxygen substituted with C₁-C₂₀ alkyl, C₁-C₂₀ acyl, C₁-C₂₀ alkoxycarbonyl, or carbamoyl.

In some methods, R¹, R² and R³ of the compound of Formula II are independently selected from a halogen atom, a hydroxyl group, an amino group, or an optionally substituted methyl group, wherein the hydrogen atoms in the methyl group are substituted with a halogen.

As used throughout, the term prodrug means a pharmacologically acceptable derivative, such as an ester or an amide, that is biotransformed in the body to form the active drug.

As used herein, the term halogen means fluorine, chlorine, bromine or iodine.

As used herein, the term aryl refers to a hydrocarbon ring system having at least one aromatic ring. Examples of aryls are phenyl, pentalenyl, indenyl, indanyl, isoindolinyl, chromanyl, naphthyl, fluorenyl, anthryl, phenanthryl and pyrenyl.

In some methods, the compound of Formula II is idoxuridine or trifluridine. Idoxuridine (Drug 1100) and trifluridine (Drug 1200) were identified as potent inducers of HbF expression in a drug screen, as described in the Examples.

In some methods, idoxuridine, an idoxuridine prodrug (for example, ropidoxuridine), trifluridine, a pharmaceutically acceptable salt of idoxuridine, a pharmaceutically acceptable salt of an idoxuridine prodrug or a pharmaceutically acceptable salt of trifluridine can be used to upregulate or increase HbF in the blood of a subject. The formulas for idoxuridine and trifluridine are set forth below.

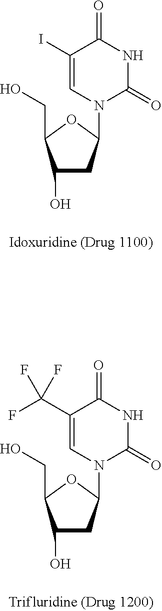 [SMILES: O=c1[nH]c(=O)n([C@H]2C[C@@H](O)[C@@H](CO)O2)cc1I ; O=c1[nH]c(=O)n([C@H]2C[C@@H](O)[C@@H](CO)O2)cc1C(F)(F)F]

The formula for an idoxuridine prodrug, i.e., ropidoxuridine, is set forth below. Ropidoxuridine is also known as 1-((2R,4S,5R)-4-hydroxy-5-(hydroxymethyl)tetrahydrofuran-2-yl)-5-iodopyrimidin-2(1H)-one.

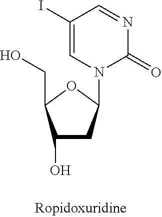 [SMILES: O=c1ncc(I)cn1[C@H]1C[C@@H](O)[C@@H](CO)O1]

Another example of an idoxuridine prodrug is 1-((2R,4S,5R)-4-hydroxy-5-(hydroxymethyl)tetrahydrofuran-2-yl)-5-iodopyri-midin-2(1H)-one (ropidoxuridine); or 4-amino-1-((2R,4S,5R)-4-hydroxy-5-(hydroxymethyl)tetrahydrofuran-2-yl)-5-iodopyrimidin-2(1H)-one (5-iodo-2′-deoxycytidine), as shown below.

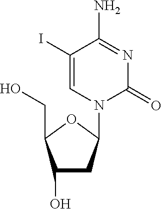 [SMILES: Nc1nc(=O)n([C@H]2C[C@@H](O)[C@@H](CO)O2)cc1I]

Other compounds that can be used to increase HbF include, but are not limited to, any of the compounds set forth in U.S. Patent Application Publication No. 20190388451.

Also provided is a method for increasing the amount of HbF in the blood of a subject, comprising administering to a subject in need thereof an effective amount of a compound having Formula I or Formula II, as defined above. In some methods, idoxuridine, an idoxuridine prodrug (for example, ropidoxuridine), trifluridine or a pharmaceutically acceptable salt thereof are administered to the subject. In some methods, idoxuridine or a pharmaceutically acceptable salt thereof, and trifluridine or a pharmaceutically acceptable salt thereof are administered to the subject. It is understood that compositions comprising the free acid, pharmaceutically acceptable salt or ester of any agent described herein that increases the amount of HbF in a subject can be administered to a subject to increase HbF expression in the blood of a subject and/or treat a hemoglobin disorder in the subject.

In some methods, the number of red blood cells increases and/or the level of HbF increases in the subject after administration of any of the compounds described herein, for example, idoxuridine, an idoxuridine prodrug, a pharmaceutically acceptable salt of idoxuridine or a pharmaceutically acceptable salt of an idoxuridine prodrug. In some methods, the number of red blood cells expressing HbF increases in the subject after administration of one or more compounds selected from the group consisting of idoxuridine, an idoxuridine prodrug, trifluridine, a pharmaceutically acceptable salt of idoxuridine, a pharmaceutically acceptable salt of trifluridine and a pharmaceutically acceptable salt of an idoxuridine prodrug. In some methods, the amount of total hemoglobin increases in the subject. Levels of HbF or total hemoglobin (including, for example, the total of HbF, hemoglobin A, hemoglobin A₂, and hemoglobin F) are detectable in biological samples of the subject, such as a sample of whole blood. The level of HbF in adults is normally less than about 0.6%. However, after administration of a compound having Formula I or Formula II, for example, idoxuridine, or a pharmaceutically acceptable salt thereof, HbF levels can be, for example, about 20-50% of total hemoglobin.

As used throughout, the terms increase, upregulate and enhanced are used interchangeably and generally mean an increase of at least 5% as compared to a reference level, for example, an increase of at least about 5%, 10%, 20%, 30%, 40%, 50%, 60%, 70%, 80%, 90%, 100%, 200%, 300%, 400% or greater as compared to a subject that has not been treated with a compound of Formula I or Formula II or that is treated with a control agent. An increase can also generally mean an increase of at least about 2-fold, 3-fold, 4-fold, 5-fold, 10-fold or greater, as compared to a reference level. An increase in HbF levels can reflect an increase in HbF expression at the mRNA and/or protein level, for example, an increase in β-globin mRNA, γ-globin mRNA, β-globin and/or γ-globin in erythropoietic cells. An increase in HbF levels can also be an increase in the percentage of HbF, an increase in the absolute amount of HbF, or an increase in the ratio of HbF:HbA in a blood sample from a subject. Optionally, after administration of a compound having Formula I or Formula II, the percentage of HbF in a blood sample of a subject is at least about 10%, 15% or 25% of the total hemoglobin present in the blood sample of the subject.

Optionally, administering an agent that increases HbF does not inhibit erythropoiesis. Optionally, administering an agent that increases HbF stimulates cell proliferation. Optionally, administering an agent that increases HbF inhibits apoptosis of erythroid progenitors (for example, hematopoietic stem cells). Optionally, an agent that increases HbF stimulates erythroid cell proliferation and survival. Optionally, administering an agent that increases HbF stimulates erythroid cell proliferation. Optionally, administering an agent that increases HbF stimulates erythroid cell survival. Optionally, administering an agent that increases HbF stimulates red blood cell production. Optionally, administering an agent that increases HbF leads to a longer survival of sickled blood cells. Optionally, administering an agent that increases HbF decreases KLF1 and/or BCL11A expression in a hemoglobin expressing cell, for example, an erythroid cell. Optionally, administering an agent that increases HbF also increases HbE.

As used throughout, the terms decrease, reduce, and inhibit are used interchangeably and generally mean an reduction of at least 5% as compared to a reference level or complete elimination, for example, a decrease of at least about 5%, 10%, 20%, 30%, 40%, 50%, 60%, 70%, 80%, 90%, 100% as compared to a subject that has not been treated or that is treated with a control agent. A decrease can also generally mean a decrease of at least about 2-fold, 3-fold, 4-fold, 5-fold, 10-fold or greater, as compared to a reference level.

As used throughout, erythropoiesis is the process that produces red blood cells (erythrocytes), i.e., the development from erythropoietic stem cell to mature red blood cell. As used throughout, the term erythropoietic cells refers to all types of nucleated cells throughout their differentiation from self-renewing hematopoietic stem cells through immature precursor cells of erythrocytes, as would be understood by persons skilled in the art. As used throughout, the term proliferation refers to an increase in a number of cells in a population of cells by means of cell division or cell renewal. Cell proliferation, for example, red blood proliferation, can also be from an increase in any erythropoietic cell.

Also provided is a method for treating a subject with a blood disorder associated with a defect in a hemoglobin producing erythropoietic cell or in the production of hemoglobin by administering to the subject with the blood disorder an effective amount of a compound having Formula I or Formula II, as defined above. In some methods, one or more compounds selected from the group consisting of idoxuridine, an idoxuridine prodrug, trifluridine or a pharmaceutically acceptable salt of idoxuridine, a pharmaceutically acceptable salt of an idoxuridine prodrug, and a pharmaceutically acceptable salt of trifluridine, are administered to the subject.

In any of the methods described herein, the subject can be a subject that has been diagnosed with a blood disorder, for example, a blood disorder associated with a defect in a hemoglobin producing erythropoietic cell or in the production of hemoglobin. Blood disorders associated with a defect in hemoglobin producing erythropoietic cells or in the production of hemoglobin include, but are not limited to, sickle cell anemia and β thalassemia.

In some methods, the number of red blood cells increases in the subject after administration of the compound of Formula I or Formula II. In some methods, the number of erythropoietic cells expressing HbF increases in the subject or in a sample from the subject after administration of the compound of Formula I or Formula II. In some methods, the amount of total hemoglobin increases in the subject after administration of the compound of Formula I or Formula II. Optionally, any of the methods described herein further comprises contacting a hemoglobin expressing cell or administering to a subject an effective amount of a second agent that increases HbF expression, i.e., an HbF inducer selected from the group consisting of hydroxyurea, a histone deacetylase (HDAC) inhibitor (for example, trichostatin), a lysine-specific histone demethylase 1 (LSD1) inhibitor (for example, tranylcypromine or RN-1), a G9a methyltransferase inhibitor (for example, UNCO638) and auranofin.

### Pharmaceutical Compositions

Pharmaceutical compositions for non-topical administration of a compound having Formula I or a pharmaceutical salt thereof, or Formula II or a pharmaceutical salt thereof, are provided herein. For example, compositions comprising idoxuridine, trifluridine, or an idoxuridine prodrug in combination with a second therapeutic agent (e.g., hydroxyurea, a histone deacetylase inhibitor, a lysine-specific histone demethylase 1 inhibitor, or a G9a methyltransferase inhibitor) are provided herein. Such pharmaceutical compositions comprise one or more effective amounts of idoxuridine, trifluridine, an idoxuridine prodrug, a pharmaceutically acceptable salt of idoxuridine, a pharmaceutically acceptable salt of trifluridine or a pharmaceutically acceptable salt of an idoxuridine prodrug, for one or more doses.

The term effective amount, as used throughout, is defined as any amount necessary to produce a desired physiologic response, for example, increasing the HbF level in a blood sample of a subject or treating a blood disorder.

Exemplary dosage amounts for administration of any compound or a pharmaceutically acceptable salt of a compound described herein include doses from about 0.1 to about 20 mg/kg of body weight of active compound per day, which may be administered in a single dose or in the form of individual divided doses, such as from 1 to 4 times per day. Alternatively, the dosage amount can be from about 0.5 to about 15 mg/kg of body weight of active compound per day, about 0.5 to 10 mg/kg of body weight of active compound per day, about 0.5 to about 5 mg/kg of body weight of active compound per day, about 0.5 to about 2.5 mg/kg of body weight of active compound per day, or from about 0.5 to about 1 mg/kg of body weight of active compound per day. Optionally, the dosage is less than about 15 mg/kg and can be less than about 14.5, 14.0, 13.5, 13.0, 12.5, 12.0, 11.5, 11.0, 10.5, 10.0, 9.5, 9.0, 8.5, 8.0, 7.5, 7.0, 6.5, 6.0, 5.5, 5.0, 4.5, 4.0, 3.5, 3.0, 2.5, 2.0, 1.5, 1.25, 1.0, 0.9, 0.8, 0.7, 0.6, 0.5, 0.4, 0.3, 0.2, mg/kg. One of skill in the art would adjust the dosage as described below based on specific characteristics of the agent and the subject receiving it.

As used herein, the term pharmaceutically acceptable salt refers to those salts that are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. Pharmaceutically acceptable salts of the compounds provided herein, for example, pharmaceutically acceptable salts of idoxuridine, or a prodrug thereof, include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, trifluoroacetic acid, undecanoate, valerate salts, and the like.

Further provided are packages or kits comprising one or more unit doses of a compound having Formula I or Formula II. For example, idoxuridine, an idoxuridine prodrug, trifluridine, a pharmaceutically acceptable salt of idoxuridine, a pharmaceutically acceptable salt of trifluridine or a pharmaceutically acceptable salt of an idoxuridine prodrug, optionally in combination with a second therapeutic agent are provided. The package can further comprise single or multiple unit doses of one or more second therapeutic agents described herein. For example, the package or kit can comprise idoxuridine, an idoxuridine prodrug, trifluridine, a pharmaceutically acceptable salt of idoxuridine, a pharmaceutically acceptable salt of trifluridine or a pharmaceutically acceptable salt of an idoxuridine prodrug, optionally in combination with a second therapeutic agent.

Effective amounts and schedules for administering one or more of the compounds described herein, for example, idoxuridine, trifluridine, an idoxuridine prodrug, a pharmaceutically acceptable salt of idoxuridine, a pharmaceutically acceptable salt of trifluridine or a pharmaceutically acceptable salt of an idoxuridine prodrug, can be determined empirically and making such determinations is within the skill in the art. The dosage ranges for administration are those large enough to produce the desired effect in which one or more symptoms of the disease or disorder are affected (e.g., reduced or delayed). The dosage should not be so large as to cause substantial adverse side effects, such as unwanted cross-reactions, unwanted cell death, and the like. Generally, the dosage will vary with the type of inhibitor, the species, age, body weight, general health, sex and diet of the subject, the mode and time of administration, rate of excretion, drug combination, and severity of the particular condition and can be determined by one of skill in the art. The dosage can be adjusted by the individual physician in the event of any contraindications. Dosages can vary and can be administered in one or more dose administrations daily.

Any of the compounds described herein can be provided in a pharmaceutical composition. These include, for example, a pharmaceutical composition comprising a therapeutically effective amount of a compound having Formula I or Formula II, for example, idoxuridine or a prodrug thereof and a pharmaceutical carrier. The term carrier means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject. Such pharmaceutically acceptable carriers include sterile biocompatible pharmaceutical carriers, including, but not limited to, saline, buffered saline, artificial cerebral spinal fluid, dextrose, and water.

Depending on the intended mode of administration, the pharmaceutical composition can be in the form of solid, semi-solid or liquid dosage forms, such as, for example, tablets, suppositories, pills, capsules, powders, liquids, or suspensions, preferably in unit dosage form suitable for single administration of a precise dosage. The compositions will include a therapeutically effective amount of the agent described herein or derivatives thereof in combination with a pharmaceutically acceptable carrier and, in addition, may include other medicinal agents, pharmaceutical agents, carriers, or diluents. By pharmaceutically acceptable is meant a material that is not biologically or otherwise undesirable, which can be administered to an individual along with the selected agent without causing unacceptable biological effects or interacting in a deleterious manner with the other components of the pharmaceutical composition in which it is contained.

As used herein, the term carrier encompasses any excipient, diluent, filler, salt, buffer, stabilizer, solubilizer, lipid, stabilizer, or other material known in the art for use in pharmaceutical formulations. The choice of a carrier for use in a composition will depend upon the intended route of administration for the composition. The preparation of pharmaceutically acceptable carriers and formulations containing these materials is described in, e.g., Remington: The Science and Practice of Pharmacy, 22nd edition, Lloyd V. Allen et al, editors, Pharmaceutical Press (2012).

Examples of physiologically acceptable carriers include buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN® (ICI, Inc.; Bridgewater, N.J.), polyethylene glycol (PEG), and PLURONICS™ (BASF; Florham Park, N.J.).

Compositions containing the agent(s) described herein suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

These compositions may also contain adjuvants such as preserving, wetting, emulsifying, and dispensing agents. Prevention of the action of microorganisms can be promoted by various antibacterial and antifungal agents, for example, parabens, chlorobutanol, phenol, sorbic acid, and the like. Isotonic agents, for example, sugars, sodium chloride, and the like may also be included. Prolonged absorption of the injectable pharmaceutical form can be brought about by the use of agents delaying absorption, for example, aluminum monostearate and gelatin.

Solid dosage forms for oral administration of the compounds described herein or derivatives thereof include capsules, tablets, pills, powders, and granules. In such solid dosage forms, the compounds described herein or derivatives thereof are admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example, paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others known in the art. They may contain opacifying agents and can also be of such composition that they release the active compound or compounds in a certain part of the intestinal tract in a delayed manner. Examples of embedding compositions that can be used are polymeric substances and waxes. The active compounds can also be in micro-encapsulated form, if appropriate, with one or more of the above-mentioned excipients.

Liquid dosage forms for oral administration of the compounds described herein or derivatives thereof include pharmaceutically acceptable emulsions, solutions, suspensions, syrups, and elixirs. In addition to the active compounds, the liquid dosage forms may contain inert diluents commonly used in the art, such as water or other solvents, solubilizing agents, and emulsifiers, such as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances, and the like.

Besides such inert diluents, the composition can also include additional agents, such as wetting, emulsifying, suspending, sweetening, flavoring, or perfuming agents.

The compositions are administered in a number of ways depending on whether local or systemic treatment is desired, and on the area to be treated. The compositions are administered via any of several routes of administration, including orally, parenterally, intravenously, intraperitoneally, intramuscularly, subcutaneously, intracavity or transdermally. Treatment may be for short periods of time or continuous throughout the lifetime of the subject. For example, the composition can be administered daily for the lifetime of the subject.

Effective doses for any of the administration methods described herein can be extrapolated from dose-response curves derived from in vitro or animal model test systems.

Throughout, treat, treating, and treatment refer to a method of reducing or delaying one or more effects or symptoms of a blood disorder, for example, a blood disorder associated with a defect in hemoglobin producing erythropoietic cells or in the production of hemoglobin. The subject can be diagnosed with a disease or disorder. Treatment can also refer to a method of reducing the underlying pathology rather than just the symptoms. The effect of the administration to the subject can have the effect of, but is not limited to, reducing one or more symptoms of the disease, a reduction in the severity of the disease, the complete ablation of the disease, or a delay in the onset or worsening of one or more symptoms. For example, a disclosed method is considered to be a treatment if there is about a 10% reduction in one or more symptoms of the disease in a subject (for example, fatigue, anemia, pain or inflammation) when compared to the subject prior to treatment or when compared to a control subject or control value. Thus, the reduction can be about a 10, 20, 30, 40, 50, 60, 70, 80, 90, 100%, or any amount of reduction in between.

As used throughout, by subject is meant an individual. The subject can be an adult subject or a pediatric subject. Pediatric subjects include subjects ranging in age from birth to eighteen years of age. Thus, pediatric subjects of less than about 10 years of age, five years of age, two years of age, one year of age, six months of age, three months of age, one month of age, one week of age or one day of age are also included as subjects. Preferably, the subject is a mammal such as a primate, and, more preferably, a human. Non-human primates are subjects as well. The term subject includes domesticated animals, such as cats, dogs, etc., livestock (for example, cattle, horses, pigs, sheep, goats, etc.) and laboratory animals (for example, ferret, chinchilla, mouse, rabbit, rat, gerbil, guinea pig, etc.). Thus, veterinary uses and medical formulations are contemplated herein.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

### Examples

### Library Screen

Briefly, for the screen, 5×10⁵ CD34⁺ cells were expanded for six days in StemSpan SFEM medium (StemCell Technologies Inc., Vancouver, Canada) with SCF, IL-3, TPO and Flt-3 ligand. After expansion, 100,000 cells were distributed to each well (in a 24-well plate) and grown in SFEM medium with 2% P/S, 20 ng/ml SCF, 1 U/ml Epo, 5 ng/ml IL-3, 2 μM dexamethasone, and 1 μM β-estradiol. Individual small molecules were added to each well. Cells were differentiated for another nine days (for RNA analysis) or fourteen days (for hemoglobin analysis). The γ- and β-globin expression in each sample were analyzed by RT-PCR or HPLC.

### Fluorescence-Activated Cell Sorting Analysis (FACS Analysis)

For fetal hemoglobin analysis, one million erythroid cells, differentiated for 14 days with or without idoxuridine in the medium, were washed with PBS that included 0.1% BSA, and fixed in 0.05% Glutaraldehyde (Sigma, G5882, St. Louis, Mo.). The fixed cells were washed three times with PBS that included 0.1% BSA, followed by permeabilization in 1% Triton X-100 (Life Technologies, HFH-10, Carlsbad, Calif.), before immunostaining with an antibody directed against HbF (Invitrogen, MHFH04, Carlsbad, Calif.).

### Western Blot Analysis

To analyze the expression of KLF1, BCL11A and LRF by Western blot, whole cell extracts were prepared from erythroid cells differentiated for seven days with DMSO or 0.7 uM of idoxuridine. Antibodies used included Actb (Santa Cruz, sc-47778 HRP, Dallas, Tex.), KLF1 (Santa Cruz, sc-14034), BCL11A (Abcam, ab19489, Cambridge, UK) and LRF (Abcam, ab175918).

### In Vivo Experiments

To test if idoxuridine can induce mouse Ey2 and human γ-globin expression in human β-BAC transgenic mice, 100 mg/kg body weight of Idoxuridine were injected intraperitoneally (IP) into mice for 4 weeks. Blood samples were obtained at the end of Idoxuridine treatment and cDNAs were synthesized. Mouse Ey2 and human γ-globin expression were analyzed by qRT-PCR.

### Drug Combinations

The combination of idoxuridine (Drug 1100) with another compound, trifluridine (Drug 1200) was tested in adult erythroid progenitors. Trifluridine reactivated γ-globin gene expression in the original screen. The erythroid progenitor cells derived from CD34+ HSPC differentiated cells in the present of idoxuridine and trifluridine were analyzed by RT-PCR for reactivated γ-globin gene expression.

### Results

Reverse transcriptase-PCR analysis showed that idoxuridine upregulates HbF expression in human CD34+-differentiated erythroid cells. γ-globin expression was significantly increased in human CD34+-differentiated erythroid cells contacted with idoxuridine.

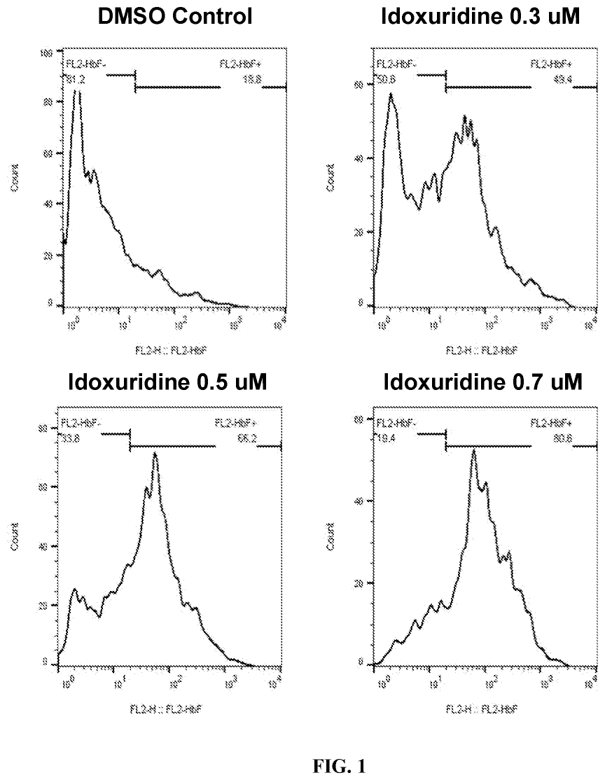

FACS analysis showed that idoxuridine increases the number of erythroid cells expressing fetal hemoglobin (F cells) in human CD34⁺-differentiated erythroid cells as compared to a DMSO control (FIG. 1).

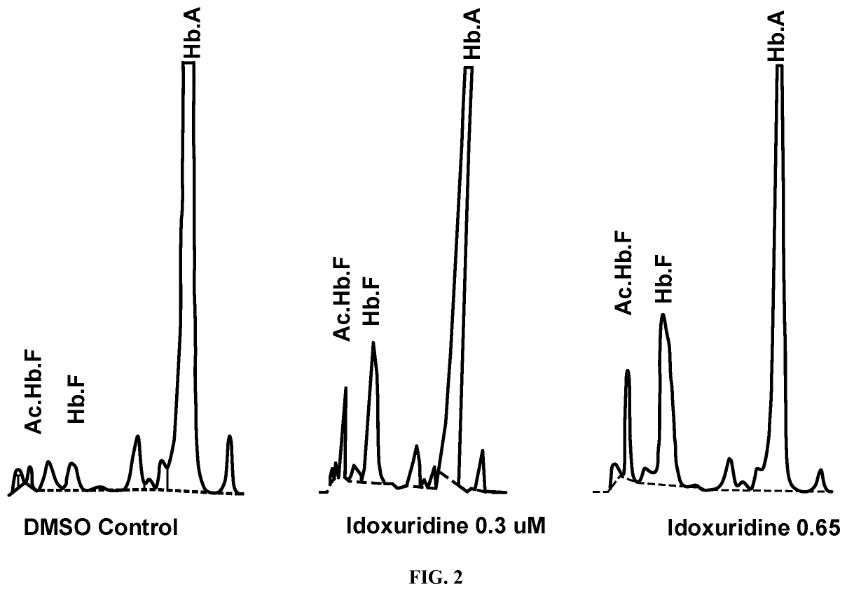

HPLC analysis showed that idoxuridine significantly increases fetal hemoglobin in human CD34⁺-differentiated erythroid cells (FIG. 2). The amount of acetylated HbF is also known.

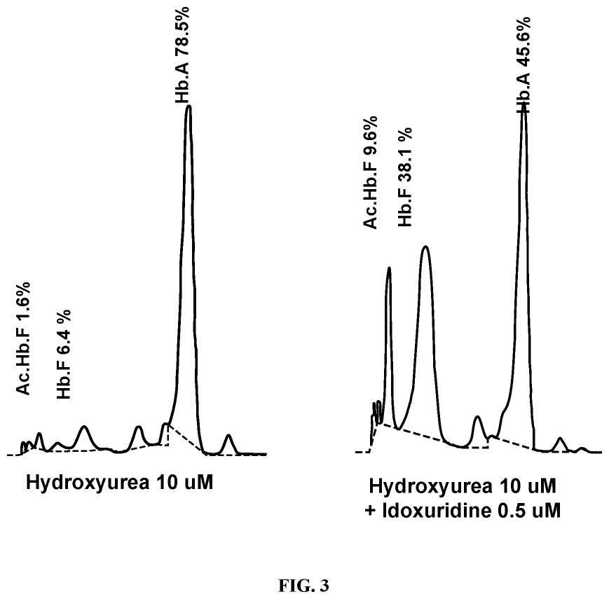

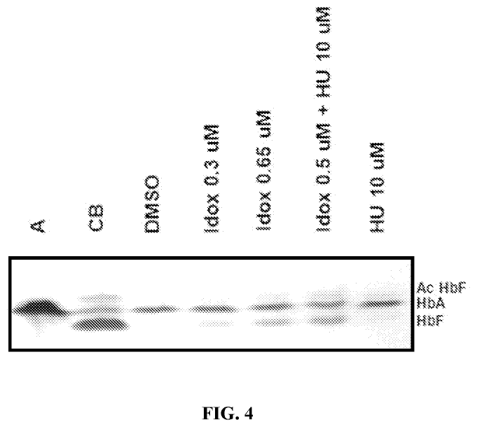

HPLC analysis showed that idoxuridine cooperates with hydroxyurea in upregulating HbF expression in human CD34⁺-differentiated erythroid cells (FIG. 3). Therefore, administration of idoxuridine and hydroxyurea results in a synergistic increase in HbF. The samples analyzed by HPLC were run on an isoelectric focusing gel. A shown in FIG. 4, idoxuridine upregulated HbF expression by human CD34⁺-differentiated erythroid cells and cooperates with hydroxyurea (HU) in upregulating HbF expression in human CD34⁺-differentiated erythroid cells, thus confirming the synergistic results obtained from the HPLC analysis.

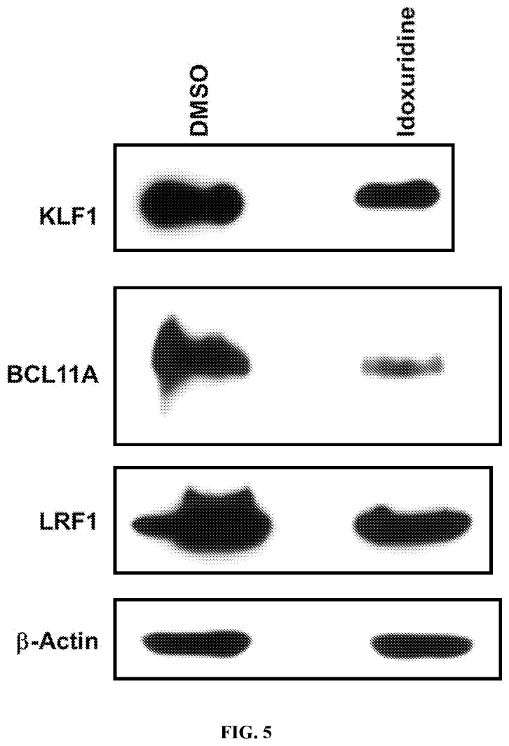

Western blot analysis showed that idoxuridine decreases KLF1 and BCL11A expression in human CD34⁺-differentiated erythroid cells (FIG. 5) and consequently upregulates γ-globin expression in adult erythroid progenitors. Interestingly, as shown in Table 1, ε-globin gene expression was also reactivated and HbE has powerful anti-sickling activity. Idoxuridine also decreased KLF1 mRNA and BCL11A mRNA expression in human CD34⁺-differentiated erythroid cells. Table 1 provides RNAseq data for human CD34⁺-differentiated erythroid cells.

TABLE 1

| Gene | DMSO Control | Idoxuridine | Fold change |
|---|---|---|---|
| SOX6 | 51 | 13 | 0.25 |
| BCL11A | 1579.8 | 478.2 | 0.303 |
| HBE1 | 30.4 | 17051 | 560 |
| HBG1 | 2296 | 18815 | 8.19 |
| HBG2 | 162 | 980 | 6 |
| HBD | 11553 | 1798 | 0.16 |
| HBB | 103638 | 20938 | 0.2 |

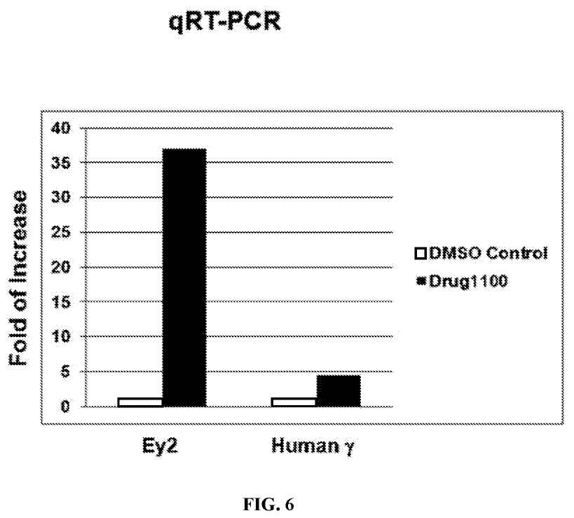

After daily intraperitoneal injections of idoxuridine for four weeks, idoxuridine increased mouse Ey-2 and human γ-globin gene expression in human β-globin BAC transgenic mice (FIG. 6).

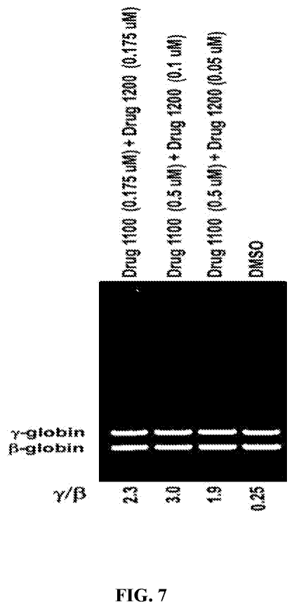

The same cDNA samples that were analyzed on the gel were quantitated by digital droplet PCR (ddPCR) to calculate the gamma- to beta-globin mRNA ratio. The RT-PCR results shown in FIG. 7 demonstrate that idoxuridine (Drug 1100) and trifluridine (Drug 1200) synergize to reactivate γ-globin gene expression in adult erythroid progenitors.

Based on these results, adult sickle pigs and Rhesus monkeys can be treated with idoxuridine and hydroxyurea, or idoxuridine and trifluridine to define γ-globin gene reactivation and HbF production. In the sickle pigs, newborns will also be treated with these drug combinations to determine whether the switch from HbF to HbS can be inhibited. Toxicity studies can also be performed. If the drugs are safe and effective, clinical trials in human adult patients to assess HbF reactivation and inhibition of sickling can be conducted. Clinical trials of human newborn sickle patients (6-9 months of age) can be performed to assess inhibition of HbF to HbS switching. If successful, this treatment would prevent tissue and organ damage before irreversible pathology occurs.

1. A method for increasing the amount of fetal hemoglobin in the blood of a subject, comprising administering to a subject in need thereof an effective amount of a compound having Formula I: 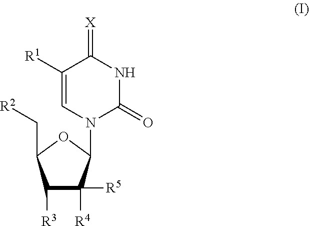
or a prodrug thereof, or a pharmaceutically acceptable salt or ester of said compound or prodrug,
wherein R¹, R² and R³ are independently a (a) halogen atom; (b) a hydroxyl group; (c) (d) an amino group; (e) a sulfhydryl group; (e) a nitro group; (f) an azido group; (g) a cyano group; (h) an ethenyl group; (i) an ethynyl group; (j) an aromatic or non-aromatic heterocyclic group; (k) an aryl group, wherein hydrogen atoms in (h), (i), (j), and (k) are optionally substituted with halogen, hydroxyl, nitro, azido, cyano, amino, sulfhydryl, phenyl, ethenyl, ethynyl, or an aromatic/non-aromatic heterocyclic group, and wherein the hydrogen atoms in the said phenyl, ethenyl, ethynyl, or aromatic/non-aromatic heterocyclic group are optionally substituted with halogen, hydroxyl, nitro, azido, cyano, amino, or sulfhydryl; (l) methyl substituted with halogen, hydroxyl, nitro, azido, cyano, amino, sulfhydryl, phenyl, ethenyl, ethynyl, or an aromatic/non-aromatic heterocyclic group, and wherein the hydrogen atoms in the said phenyl, ethenyl, ethynyl, and aromatic/non-aromatic heterocyclic group are optionally substituted with halogen, hydroxyl, nitro, azido, cyano, amino, or sulfhydryl; or (m) oxygen substituted with C₁-C₂₀ alkyl, C₁-C₂₀ acyl, C₁-C₂₀ alkoxycarbonyl, or carbamoyl;
X is O or S;
Y is H, O, S, or N; and
R⁴ and R⁵ are independently H, —OH (hydroxyl), halogen, or C₁-C₂₀ alkyl.

2. The method of claim 1, wherein the compound has Formula II 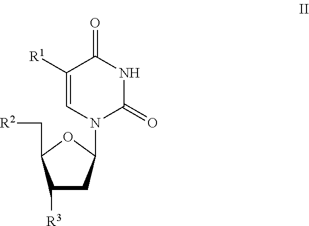
or a prodrug thereof, or a pharmaceutically acceptable salt or ester of said compound or prodrug,
wherein R¹, R² and R³ are independently (a) a halogen atom; (b) a hydroxyl group; (c) an amino group; (d) a sulfhydryl group; (e) a nitro group; (f) an azido group; (g) a cyano group; (h) an ethenyl group; (i) an ethynyl group; (j) an aromatic/non-aromatic heterocyclic group; (k) an aryl group, wherein hydrogen atoms in (h), (i), (j) and (k) are optionally substituted with halogen, hydroxyl, nitro, azido, cyano, amino, sulfhydryl, phenyl, ethenyl, ethynyl, or an aromatic/non-aromatic heterocyclic group, and wherein the hydrogen atoms in the said phenyl, ethenyl, ethynyl, or aromatic/non-aromatic heterocyclic group are optionally substituted with halogen, hydroxyl, nitro, azido, cyano, amino, or sulfhydryl; (l) methyl substituted with halogen, hydroxyl, nitro, azido, cyano, amino, sulfhydryl, phenyl, ethenyl, ethynyl, or an aromatic/non-aromatic heterocyclic group, and wherein the hydrogen atoms in the said phenyl, ethenyl, ethynyl, and aromatic/non-aromatic heterocyclic group are optionally substituted with halogen, hydroxyl, nitro, azido, cyano, amino, or sulfhydryl; or (m) oxygen substituted with C₁-C₂₀ alkyl, C₁-C₂₀ acyl, C₁-C₂₀ alkoxycarbonyl, or carbamoyl.

3. The method of claim 1, wherein the compound is selected from the group consisting of idoxuridine, an idoxuridine prodrug, trifluridine or a pharmaceutically acceptable salt of idoxuridine, a pharmaceutically acceptable salt of an idoxuridine prodrug, and a pharmaceutically acceptable salt of trifluridine.

4. The method of claim 3, wherein the compound is idoxuridine, a prodrug thereof, or a pharmaceutically acceptable salt thereof.

5. The method of claim 1, wherein idoxuridine or a pharmaceutically acceptable salt thereof, and trifluridine or a pharmaceutically acceptable salt thereof are administered to the subject.

6. The method of claim 1, wherein the method further comprises administering to the subject an effective amount of a fetal hemoglobin inducer selected from the group consisted of hydroxyurea, a histone deacetylase (HDAC) inhibitor, a lysine-specific histone demethylase 1 (LSD1) inhibitor and a G9a methyltransferase inhibitor.

7. The method of claim 1, wherein the subject has been diagnosed with a blood disorder.

8. The method of claim 7, wherein the blood disorder is a blood disorder associated with a defect in hemoglobin producing erythropoietic cells or in the production of hemoglobin.

9. The method of claim 8, wherein the blood disorder is selected from the group consisting of sickle cell anemia or β thalassemia.

10. The method of claim 1, wherein the number of red blood cells increases in the subject after administration of the compound.

11. The method of claim 10, wherein the number of erythropoietic cells expressing fetal hemoglobin increases in the subject after administration of the idoxuridine.

12. The method of claim 1, wherein the amount of total hemoglobin increases in the subject after administration of the compound.

13. The method of claim 1, wherein the subject is a human subject.

14. The method of claim 13, wherein the human subject is a pediatric subject.

15. The method of claim 1, wherein the compound is administered at a dosage of between about 1 mg/kg and about 10 mg/kg.

16. The method of claim 1, wherein the compound is administered orally or intravenously.

17. A method for treating a subject with a blood disorder associated with a defect in hemoglobin producing erythropoietic cells or in the production of hemoglobin, comprising administering to the subject with the blood disorder an effective amount of an effective amount of a compound having Formula I: 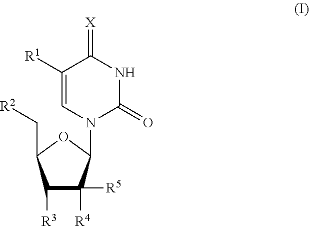
or a prodrug thereof, or a pharmaceutically acceptable salt or ester of said compound or prodrug,
wherein R¹, R² and R³ are independently a (a) halogen atom; (b) a hydroxyl group; (c) (d) an amino group; (e) a sulfhydryl group; (e) a nitro group; (f) an azido group; (g) a cyano group; (h) an ethenyl group; (i) an ethynyl group; (j) an aromatic or non-aromatic heterocyclic group; (k) an aryl group, wherein hydrogen atoms in (h), (i), (j), and (k) are optionally substituted with halogen, hydroxyl, nitro, azido, cyano, amino, sulfhydryl, phenyl, ethenyl, ethynyl, or an aromatic/non-aromatic heterocyclic group, and wherein the hydrogen atoms in the said phenyl, ethenyl, ethynyl, or aromatic/non-aromatic heterocyclic group are optionally substituted with halogen, hydroxyl, nitro, azido, cyano, amino, or sulfhydryl; (l) methyl substituted with halogen, hydroxyl, nitro, azido, cyano, amino, sulfhydryl, phenyl, ethenyl, ethynyl, or an aromatic/non-aromatic heterocyclic group, and wherein the hydrogen atoms in the said phenyl, ethenyl, ethynyl, and aromatic/non-aromatic heterocyclic group are optionally substituted with halogen, hydroxyl, nitro, azido, cyano, amino, or sulfhydryl; or (m) oxygen substituted with C₁-C₂₀ alkyl, C₁-C₂₀ acyl, C₁-C₂₀ alkoxycarbonyl, or carbamoyl;
X is O or S;
Y is H, O, S, or N; and
R⁴ and R⁵ are independently H, —OH (hydroxyl), halogen, or C₁-C₂₀ alkyl.

18. The method of claim 17, wherein the compound has Formula II 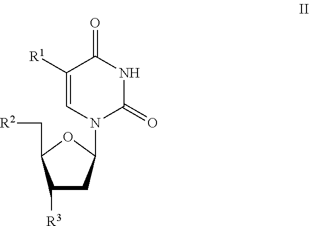
or a prodrug thereof, or a pharmaceutically acceptable salt or ester of said compound or prodrug,
wherein R¹, R² and R³ are independently (a) a halogen atom; (b) a hydroxyl group; (c) an amino group; (d) a sulfhydryl group; (e) a nitro group; (f) an azido group; (g) a cyano group; (h) an ethenyl group; (i) an ethynyl group; (j) an aromatic/non-aromatic heterocyclic group; (k) an aryl group, wherein hydrogen atoms in (h), (i), (j) and (k) are optionally substituted with halogen, hydroxyl, nitro, azido, cyano, amino, sulfhydryl, phenyl, ethenyl, ethynyl, or an aromatic/non-aromatic heterocyclic group, and wherein the hydrogen atoms in the said phenyl, ethenyl, ethynyl, or aromatic/non-aromatic heterocyclic group are optionally substituted with halogen, hydroxyl, nitro, azido, cyano, amino, or sulfhydryl; (l) methyl substituted with halogen, hydroxyl, nitro, azido, cyano, amino, sulfhydryl, phenyl, ethenyl, ethynyl, or an aromatic/non-aromatic heterocyclic group, and wherein the hydrogen atoms in the said phenyl, ethenyl, ethynyl, and aromatic/non-aromatic heterocyclic group are optionally substituted with halogen, hydroxyl, nitro, azido, cyano, amino, or sulfhydryl; or (m) oxygen substituted with C₁-C₂₀ alkyl, C₁-C₂₀ acyl, C₁-C₂₀ alkoxycarbonyl, or carbamoyl.

19. The method of claim 17, wherein the compound is selected from the group consisting of idoxuridine, an idoxuridine prodrug, trifluridine or a pharmaceutically acceptable salt of idoxuridine, a pharmaceutically acceptable salt of an idoxuridine prodrug, and a pharmaceutically acceptable salt of trifluridine.

20. The method of claim 19, wherein the compound is idoxuridine, a prodrug thereof, or a pharmaceutically acceptable salt thereof.

21. The method of claim 17, wherein idoxuridine or a pharmaceutically acceptable salt thereof, and trifluridine or a pharmaceutically acceptable salt thereof are administered to the subject.

22. The method of claim 17, wherein the method further comprises administering to the subject an effective amount of a fetal hemoglobin inducer selected from the group consisted of hydroxyurea, a histone deacetylase (HDAC) inhibitor, a lysine-specific histone demethylase 1 (LSD1) inhibitor and a G9a methyltransferase inhibitor.

23. The method of claim 17, wherein the blood disorder is selected from the group consisting of sickle cell anemia or β thalassemia.

24. The method of claim 23, wherein the number of red blood cells increases in the subject.

25. The method of claim 24, wherein the number of erythropoietic cells expressing fetal hemoglobin increases in the subject after administration of the compound.

26. The method of claim 17, wherein the amount of total hemoglobin increases in the subject after administration of the compound.

27. The method of claim 17, wherein the subject is a human subject.

28. The method of claim 27, wherein the human subject is a pediatric subject.

29. The method of claim 17, wherein the compound is administered at a dosage of between about 1 mg/kg and about 10 mg/kg.

30. The method of claim 17, wherein the compound is administered orally or intravenously.
